(19) United States
(12) Patent Application Publication
(10) Pub. No.: US 2011/0218190 A1
(43) Pub. Date: Sep. 8, 2011

(54) THERAPEUTIC USES OF AMPA RECEPTOR MODULATORS FOR TREATMENT OF MOTOR DYSFUNCTION

(75) Inventors: Walter E. Babiec, Palos Verdes Peninsula, CA (US); Shane A. Saywell, Santa Monica, CA (US); Jack L. Feldman, Sherman Oaks, CA (US); Wiktor A. Janczewski, Los Angeles, CA (US)

(73) Assignee: THE REGENTS OF THE UNIVERSITY OF CALIFORNIA, Oakland, CA (US)

(21) Appl. No.: 13/128,326

(22) PCT Filed: Nov. 9, 2009
(86) PCT No.: PCT/US09/63758

§ 371 Date: May 9, 2011

Related U.S. Application Data
(60) Provisional application No. 61/113,173, filed on Nov. 10, 2008.

(51) Int. Cl. A61K 31/549; A61K 31/453; A61K 31/4015; A61P 11/00

(52) U.S. Cl. 514/223.2; 514/321; 514/423

(57) ABSTRACT

The application describes treatment of motoneuronal dysfunctions or disorders using AMPA receptor modulators. Examples of a motoneuronal dysfunction or disorder include obstructive sleep apnea, snoring, multiple sclerosis, spinal cord injury, e.g., crush, partial or complete transection, motor neuron diseases, motor weakness due to aging, stroke, tumor, hemorrhage, degenerative or wasting diseases, and spasticity. One example of an AMPA receptor modulator is a benzothiadiazide compound. Another example of an AMPA receptor modulator is an ampakine. Yet another example of an AMPA receptor modulator is aniracetam.

### CROSS-REFERENCE TO RELATED APPLICATIONS

This application claims the benefit under 35 U.S.C. §119(e) of U.S. Provisional Application No. 61/113,173, filed 10 Nov. 2008, the entirety of which is hereby incorporated by reference.

### STATEMENT REGARDING FEDERALLY SPONSORED R&D

This invention was made with Government support of Grant No. NS024742, awarded by the National Institutes of Health. The Government has certain rights in this invention.

### BACKGROUND OF THE INVENTION

1. Field of the Invention

Methods relating to treatment of motor dysfunctions or disorders are provided. In particular, methods relating to the use two classes of drugs, (i) benzothiadiazides, (ii) ampakines and their structural antecedents, e.g., aniracetam, are provided. Both classes of drugs are considered to be modulators of α-amino-3-hydroxy-5-methyl-4-isoxazolepropionic acid (AMPA) receptor deactivation and desensitization in the nervous system.

2. Background

### A. Sleep-Disordered Breathing

Increasingly, sleep-disordered breathing is recognized clinically as a spectrum of disorders with serious health consequences. Within this spectrum two of the most common conditions are snoring and obstructive sleep apnea (OSA). Snoring has many causes, one of which includes flaccidity of the upper airway. OSA is a disorder of repetitive upper airway collapse during sleep. OSA involves the loss of tone in muscles controlling upper airway resistance, including and often primarily the genioglossus (GG) muscle of the tongue, which is innervated by the medial branch of the hypoglossal nerve. These collapses cause sufferers to stop breathing (apnea), often for a minute or longer and as many as hundreds of times per night. As many as 1 in 5 adults have at least mild OSA whereas at least 2% of women and 4% of men have clinically significant (moderate to severe) OSA.

OSA is associated with a variety of health risks. Apneic events induce central hypoxia, which leads to activation of the sympathetic nervous system, resulting in acute hypertension and tachycardia. Additionally, activation of the sympathetic nervous system leads to arousals from sleep, resulting in daytime fatigue, often with serious consequences, for example, falling asleep when driving. Chronically, repeated hypoxia and spiking blood pressure cause increased propensity for neurocognitive impairment, hypertension, myocardial infarction and stroke. The economic burden of OSA in the US is estimated to be several billions of dollars annually.

Chronic snoring often predicts the development of OSA. Although snoring is a symptom for those who suffer from OSA, not all people who snore have OSA. Moreover, not all people who snore develop OSA. Snoring is far more prevalent, present at various levels in 30% to 50% of the general adult population. Habitual snoring has been estimated to be prevalent in at least 20% of the male population. Snoring often disrupts the sufferer's sleep and that of their bed fellows. Like OSA, snoring is associated with daytime fatigue, decreased work productivity, and increased risk for occupational accidents.

The genioglossus muscle of the tongue contributes to upper airway patency. Proper functioning of the genioglossus muscle helps prevent partial or complete closure of the upper airway. The hypoglossal (XII) nerve, which originates from the hypoglossal motor nucleus in the medulla, innervates muscles of the upper airway including the genioglossus muscle. Genioglossus muscle activity increases tongue rigidity during inspiration. During sleep, however, genioglossus activity is reduced. In OSA patients, this produces muscle flaccidity in the genioglossus and other upper airway muscles severe enough to occlude or significantly reduce air flow during inhalation. Similar to OSA, the pathophysiological mechanism(s) underlying snoring is not well understood.

Current interventions for sleep apnea include machines providing continuous positive airway pressure (CPAP), intrusive dental appliances, or reconstructive surgery to reshape the soft pallet and other parts of the patient's upper airway to reduce obstructions. However, CPAP and appliances typically have a low level of patient compliance. In the long-term, surgery is often ineffective. PCT Int'l Pub. No. WO 08025148 describes use of a drug, the ampakine CX546, to inhibit respiratory depression caused by opiates or barbiturates.

Current interventions for snoring also include intrusive dental appliances and surgery. For instance, the genioglossal advancement procedure for treating OSA or snoring involves surgical repositioning of the tongue by cutting a window in the lower jaw, moving the bone where the tongue attaches forward, and reattaching the bone to the lower jaw with screws. These treatments are invasive, suffer from low compliance, and over time surgery is often ineffective. These invasive and expensive interventions present a need to increase upper airway muscle tone through pharmaceutical intervention.

### B. Multiple Sclerosis

Multiple sclerosis (MS) is a disease of demyelination and degeneration, which is the result of a harmful episodic or continuous autoimmune response. During outbreaks of the disease, the myelin surrounding nerves breaks down and reduces the effectiveness of neural transmission to the musculature. Outbreaks can result in cognitive impairment. Pharmaceutical treatments for motor and cognitive deficiencies resulting from episodic outbreaks are limited.

### C. Spinal Cord Injury

Spinal cord injury (SCI), due to crush, partial or complete transection, tumor, operative trauma, and ischemic injury, e.g., stroke, often result in partial or total loss of sensory or motor function to areas of the body served by the spinal cord above and below the injury or parts of the body served by the brain area that suffered the stroke. Scientists are studying protocols for restoring movement following SCI that involve treatment with neuromodulator agonists for neurotransmitters such as serotonin, norepinephrine, and dopamine in combination with physical training and electrical stimulation. Yuri, P., et al. (2007) J Neurophysiol 98:2525-36. Pharmaceutical treatments to improve sensory or motor function in sufferers of mild to severe spinal cord injury are limited.

### SUMMARY OF THE INVENTION

Provided herein are methods of treating motor dysfunctions and disorders and therapeutic uses of AMPA receptor modulators.

One embodiment is the use of an AMPA receptor modulator in the manufacture of a medicament for increasing genioglossus muscle tone in a subject. In one aspect of the embodiment, the AMPA receptor modulator is an ampakine, such as CX546. In the same aspect, genioglossus muscle activity is increased by at least 15%. Also in the same aspect, the genioglossus muscle activity is increased for at least two hours. Further in the same aspect, the effective amount of the ampakine sufficient to increase genioglossus muscle activity is also sufficient to reduce or inhibit snoring in the subject.

In another aspect of the embodiment, the AMPA receptor modulator is a benzothiadiazide, such as cyclothiazide. In the same aspect, genioglossus muscle activity is increased by at least 50%. Further in the same aspect, genioglossus muscle activity is increased for at least two hours. In another aspect, the effective amount of the benzothiadiazide sufficient to increase genioglossus muscle activity is also sufficient to reduce or inhibit obstructive sleep apnea in the subject. In another aspect, the effective amount of the benzothiadiazide sufficient to increase genioglossus muscle activity is also sufficient to reduce or inhibit snoring in the subject.

In a further aspect of the preceding embodiment, the AMPA receptor modulator is aniracetam. In the same aspect, the effective amount of the aniracetam sufficient to increase genioglossus muscle activity is also sufficient to reduce or inhibit snoring in the subject. In another aspect, the effective amount of aniracetam sufficient to increase genioglossus muscle activity is also sufficient to reduce or inhibit obstructive sleep apnea in the subject.

One embodiment is the use of an AMPA receptor modulator in the manufacture of a medicament for the treatment of snoring in a subject. In one aspect, the AMPA receptor modulator is an ampakine, such as CX546. In another aspect, the AMPA receptor modulator is a benzothiadiazide, such as cyclothiazide. In a further aspect, the AMPA receptor modulator is aniracetam.

One embodiment is the use of a benzothiadiazide in the manufacture of a medicament for the treatment of obstructive sleep apnea. In one aspect, the benzothiadiazide is cyclothiazide.

One embodiment is the use of a benzothiadiazide in the manufacture of a medicament for the treatment of multiple sclerosis in a subject. In one aspect, the benzothiadiazide is cyclothiazide.

One embodiment is the use of a benzothiadiazide in the manufacture of a medicament for the treatment of spinal cord injury in a subject. In one aspect, the benzothiadiazide is cyclothiazide. In another aspect, the spinal cord injury is caused by crush, partial or complete transection, tumor, trauma, or ischemic injury. In the same aspect, ischemic injury is stroke.

One embodiment is the use of aniracetam in the manufacture of a medicament for the treatment of obstructive sleep apnea.

One embodiment is the use of aniracetam in the manufacture of a medicament for the treatment of multiple sclerosis in a subject.

One embodiment is the use of aniracetam in the manufacture of a medicament for the treatment of spinal cord injury in a subject. In one aspect, the spinal cord injury is caused by crush, partial or complete transection, tumor, trauma, or ischemic injury. In the same aspect, ischemic injury is stroke.

One embodiment is a method of increasing genioglossus muscle tone in a subject including administering to the subject an effective amount of an AMPA receptor modulator sufficient to increase genioglossus activity.

In one aspect of the embodiment, the AMPA receptor modulator is an ampakine, such as CX546 for example. In the same aspect, the genioglossus muscle is increased by at least 15%. Also in the same aspect, the genioglossus muscle activity is increased for at least two hours. Further in the same aspect, the effective amount of the ampakine sufficient to increase genioglossus muscle activity is also sufficient to reduce or inhibit snoring in the subject.

In another aspect of the embodiment, the AMPA receptor modulator is a benzothiadiazide, such as cyclothiazide. In the same aspect, the genioglossus muscle is increased by at least 50%. Also in the same aspect, the genioglossus muscle activity is increased for at least two hours. In another aspect, the effective amount of the benzothiadiazide sufficient to increase genioglossus muscle activity is also sufficient to reduce or inhibit snoring in the subject. In yet another aspect, the effective amount of the benzothiadiazide sufficient to increase genioglossus muscle activity is also sufficient to reduce or inhibit obstructive sleep apnea in the subject.

In a further aspect of the preceding embodiment, the AMPA receptor modulator is aniracetam. In the same aspect, the effective amount of the aniracetam sufficient to increase genioglossus muscle activity is also sufficient to reduce or inhibit snoring in the subject. In another aspect, the effective amount of aniracetam sufficient to increase genioglossus muscle activity is also sufficient to reduce or inhibit obstructive sleep apnea in the subject.

One embodiment is a method of treating snoring in a subject including administering to the subject an effective amount of an AMPA receptor modulator sufficient to reduce or inhibit snoring. In one aspect, the AMPA receptor modulator is an ampakine, such as CX546. In another aspect, the AMPA receptor modulator is a benzothiadiazide, such as cyclothiazide. In a further aspect, the AMPA receptor modulator is aniracetam.

One embodiment is a method of treating obstructive sleep apnea in a subject including administering to the subject an effective amount of a benzothiadiazide sufficient to reduce or inhibit obstructive sleep apnea. In one aspect, the benzothiadiazide is cyclothiazide.

One embodiment is a method of treating obstructive sleep apnea in a subject including administering to the subject an effective amount of aniracetam sufficient to reduce or inhibit obstructive sleep apnea.

One embodiment is a method of treating multiple sclerosis in a subject including administering to the subject an effective amount of an AMPA receptor modulator sufficient to improve motor or cognitive deficiency in the subject resulting from multiple sclerosis. In one aspect, the AMPA receptor modulator is an ampakine, such as CX546. In another aspect, the AMPA receptor modulator is a benzothiadiazide, such as cyclothiazide. In a further aspect, the AMPA receptor modulator is aniracetam.

One embodiment is a method of treating spinal cord injury in a subject including administering to the subject an effective amount of an AMPA receptor modulator sufficient to improve sensory or motor function in the subject. In one aspect, the spinal cord injury is caused by crush, partial or complete transection, tumor, trauma, or ischemic injury. In such an aspect, the ischemic injury is stroke. In another aspect, the AMPA receptor modulator is an ampakine, such as CX546. In yet another aspect, the AMPA receptor modulator is a benzothiadiazide, such as cyclothiazide. In a further aspect, the AMPA receptor modulator is aniracetam.

One embodiment is a kit for treating snoring in a subject including a pharmaceutical preparation of an AMPA receptor modulator and instructions for use of the pharmaceutical preparation to treat snoring. In one aspect, the AMPA receptor modulator is an ampakine, such as CX546. In another aspect, the AMPA receptor modulator is a benzothiadiazide, such as cyclothiazide. In yet another aspect, AMPA receptor modulator is aniracetam.

One embodiment is a kit for treating obstructive sleep apnea in a subject including a pharmaceutical preparation of an AMPA receptor modulator and instructions for use of the pharmaceutical preparation to treat obstructive sleep apnea. In one aspect, the AMPA receptor modulator is an ampakine, such as CX546. In another aspect, the AMPA receptor modulator is a benzothiadiazide, such as cyclothiazide. In yet another aspect, AMPA receptor modulator is aniracetam.

One embodiment is a kit for treating multiple sclerosis in a subject including a pharmaceutical preparation of an AMPA receptor modulator and instructions for use of the pharmaceutical preparation to treat multiple sclerosis. In one aspect, the AMPA receptor modulator is an ampakine, such as CX546. In another aspect, the AMPA receptor modulator is a benzothiadiazide, such as cyclothiazide. In yet another aspect, AMPA receptor modulator is aniracetam.

One embodiment is a kit for treating spinal cord injury in a subject including a pharmaceutical preparation of an AMPA receptor modulator and instructions for use of the pharmaceutical preparation to treat obstructive spinal cord injury. In one aspect, the AMPA receptor modulator is an ampakine, such as CX546. In another aspect, the AMPA receptor modulator is a benzothiadiazide, such as cyclothiazide. In yet another aspect, AMPA receptor modulator is aniracetam.

### BRIEF DESCRIPTION OF THE DRAWINGS

FIGS. 1A and 1B are graphs showing the effects on endogenous integrated hypoglossal (XII) nerve recordings following bath application of cyclothiazide (CTZ) (FIG. 1A) and CX546 (FIG. 1B) to the in vitro neonatal rat medullary slice preparation.

FIG. 2 is a line graph of group data showing percent increase of integrated hypoglossal (XII) amplitude following short-duration application of cyclothiazide or CX546.

FIG. 3 is a line graph of group data showing percent increase of integrated hypoglossal (XII) amplitude in a cyclothiazide dose-dependent manner.

FIG. 4A is a graph showing hypoglossal (XII) nerve burst and associated XII motoneuron drive currents from periods of pre-treatment control and 1 hour post-treatment with cyclothiazide. FIG. 4B is a bar graph of group data comparing hypoglossal (XII) motoneuron charge transfer and integrated XII peak amplitude between periods of pre-treatment control and 1 hour post-treatment with cyclothiazide.

FIGS. 5A and 5B are graphs showing the effects on endogenous, integrated hypoglossal (XII) nerve activity of bath application of low doses of CX546 (FIG. 8A) or aniracetam (FIG. 8B) to the in vitro neonatal rat medullary slice preparation.

FIGS. 6A and 6B are graphs showing integrated genioglossus (GG) muscle activity and respiratory rate in an anesthetized, freely breathing rat prior to application (FIG. 6A) and 8 minutes after applying (FIG. 6B) cyclothiazide to the hypoglossal (XII) motor nucleus.

FIGS. 7A and 7B are graphs showing integrated genioglossus (GG) muscle activity and respiratory rate in an anesthetized, freely breathing rat during control (FIG. 7A) and 30 minutes after applying cyclothiazide (FIG. 7B) to the hypoglossal (XII) motor nucleus.

FIG. 8 is a graph showing integrated genioglossus (GG) muscle output (FIG. 8A) and respiratory rate (FIG. 8B) in an anesthetized, freely breathing rat prior to application and after applying cyclothiazide to the IV^{th} ventricle.

FIG. 9 is a graph showing integrated genioglossus (GG) muscle output (FIG. 9A) and respiratory rate (FIG. 9B) after injecting CX546 into the femoral vein of an anesthetized, freely behaving rat.

### DETAILED DESCRIPTION

### I. Introduction

Disclosed herein are several embodiments for treating motor dysfunctions or disorders including, for example, obstructive sleep apnea (OSA), snoring, multiple sclerosis (MS), and spinal cord injury (SCI). Various embodiments of compositions and methods of treating motor dysfunctions or disorders including, for example, obstructive sleep apnea, snoring, multiple sclerosis, spinal cord injury, e.g., crush, partial or complete transection, motor neuron diseases, motor weakness due to aging, stroke, tumor, hemorrhage, degenerative or wasting diseases, and spasticity are disclosed. A variety of embodiments described relate to increasing genioglossus muscle tone by administering AMPA receptor modulator(s) to a subject. A variety of embodiments relate to a pharmacological treatment that facilitate respiratory output to the upper airway muscles to reduce or inhibit loss of muscle tone during sleep. In addition several embodiments relate to a pharmacological treatment that facilitates respiratory output to other muscle groups.

A. Neural Control of Breathing

Motoneurons are the ultimate arbiters of movement. In the case of breathing, movements are initiated by a central pattern generator, the preBötzinger Complex (preBötC), located in the medulla, and transmitted to the motoneurons via interneuronal networks. These interneurons transmit respiratory drive to motoneurons innervating respiratory muscles, e.g., the upper airway, including the genioglossus muscle, diaphragm, intercostals and abdominal muscles. The respiratory drive to the motoneurons is largely mediated via α-amino-3-hydroxy-5-methyl-4-isoxazolepropionic acid (AMPA) receptors.

B. Spinal Cord Injury

Injury to the spinal cord can occur due to a wide variety of traumatic events. For example, spinal cord injury (SCI) can be due to crush, partial or complete transection, tumor, operative trauma, or ischemic injury, e.g., stroke, often resulting in partial or total loss of sensory or motor function to areas of the body served by the spinal cord above and below the injury or parts of the body served by the brain area that suffered the stroke.

One consequence of the injury underlying loss of motor function due to SCI is damage to the long descending tracts from the brain that send the motor command to the segmental pattern generator (including the motoneurons) in the spinal cord. However, in the majority of patients with SCI these tracts retain some integrity and thus residual function is often present.

Glutamate is an excitatory transmitter mediating descending motor commands that acts on AMPA receptors at the level of the motoneuron. Potentiation of the descending drive by modulating AMPA receptor properties can increase the output of the motoneurons, potentially restoring some degree of motor function.

C. Diseases of Demyelination

The mature central and peripheral nervous systems rely on the myelination of many of their axons to (i) increase the speed and distance that action potentials travel and (ii) increase the axonal packing density of nerves by allowing the use of thinner axons (Koch, C. (1999) Biophysics of Computation, Oxford University Press: New York). The myelin, which is formed by oligodendrocytes and Schwann cells in the central and peripheral nervous systems respectively, relies upon myelin basic proteins for proper compaction of the myelin around the axons. Experimentally, injection of these proteins into mammals causes local inflammation and destruction of myelin sheaths. This is thought to mirror the pathology underlying diseases of demyelination, for example multiple sclerosis, which are considered to be autoimmune diseases (Kandel, E. R., et al. (2009) Principles of Neuroscience, McGraw-Hill: New York). Multiple sclerosis, therefore, by way of causing demyelination, severely impairs neural circuits by degrading neural transmission.

D. Allosteric Modulators of AMPA Receptor Deactivation and Desensitization

Not wishing to be bound to theory, AMPA receptor modulators that reduce receptor desensitization and delay deactivation are compounds, that generally are believed to bind to AMPA receptors to make the receptor conformation resistant to desensitization or deactivation. Binding of these anti-deactivation/anti-desensitization agents to the receptor, increases the receptor affinity for its agonist, resulting in the channel remaining in the open, (Arai, A. C. and Kessler, M. (2007) Curr. Drug Targets 8(5):583-602).

It will be appreciated by one of skill in the art that AMPA receptor modulators can have other pharmacological properties in addition to those associated with allosteric modulation of AMPA receptor deactivation and desensitization, which may also affect AMPA receptor activity. For example stimulation of BDNF synthesis (Lauterborn, J. C. et al. (2003) J. Pharmacol. Exp. Ther. 307(1):297-305). Hence, an AMPA receptor modulator can have a plurality of pharmacological properties through a variety of mechanisms or pathways that are not mutually exclusive.

Non-limiting examples of AMPA receptor modulators suitable for use in the practice of the embodiments described herein are disclosed in PCT Int'l Pub. No. WO 9402475 and in related U.S. Pat. Nos. 5,773,434; 5,488,049; 5,650,409; 5,736,543; 5,747,492; 5,773,434; 5,891,876; 6,030,968; 6,274,600; 6,329,368; 6,943,159; 7,026,475; and U.S. Pat. Pub. No. 20020055508. Further non-limiting examples of AMPA receptor modulators suitable for use with the embodiments described herein include: sulfonamide derivatives as disclosed in U.S. Pat. Nos. 6,174,922; 6,303,816; 6,358,981; 6,362,230; 6,500,865; 6,515,026; 6,552,086; PCT Int'l Pub. Nos. WO 0190057, WO 0190056, WO 0168592, WO 0196289, WO 02098846, WO 0006157, WO 9833496, WO 0006083, WO 0006148, WO 0006149, WO 9943285, WO 9833496; (bis)sulfonamide derivatives as disclosed in WO 0194306; N-substituted sulfonamide derivatives as disclosed in U.S. Pat. No. 6,525,099 and PCT Int'l Pub. No. WO 0006537; heterocyclic sulfonamide derivatives as disclosed in U.S. Pat. No. 6,355,655 and PCT Int'l Pub. Nos. WO 0214294, WO 0214275, and WO 0006159; heterocyclyl sulfonamide derivatives as disclosed in U.S. Pat. No. 6,358,982 and PCT Int'l Pub. No. WO 0006158; alkenyl sulfonamide derivatives as disclosed in U.S. Pat. No. 6,387,954 and PCT Int'l Pub. No. WO 0006539; cycloalkenyl sulfonamide derivatives as disclosed in PCT Int'l Pub. No. WO 02098847; cyclopentyl sulfonamide derivatives as disclosed in U.S. Pat. No. 6,639,107 and PCT Int'l Pub. No. WO 0142203; cycloalkylfluoro sulfonamide derivatives as disclosed in PCT Int'l Pub. No. WO 0232858; acetylenic sulfonamide derivatives as disclosed in PCT Int'l Pub. No. WO0218329; 2-propane-sulfonamide compounds and derivatives as disclosed in U.S. Pat. No. 6,596,716 and PCT Int'l Pub. Nos: WO 06087169, WO 06015827, WO 06015828, WO 06015829, WO 07090840, and WO 07090841; and 2-aminobenzenesulfonamide derivatives as disclosed in WO 02089734.

Further non-limiting examples of AMPA receptor modulators suitable for use with the embodiments described herein include: benzoyl piperidine, benzoyl derivatives, and pyrrolidine compounds and related structures as disclosed in U.S. Pat. Nos. 5,650,409; 5,747,492; 5,783,587; 5,852,008; and 6,274,600; compounds based on benzoxazine ring systems as disclosed in U.S. Pat. Nos. 5,736,543; 5,962,447; 5,985,871; and PCT Int'l Pub. Nos. WO 9736907 and WO 9933469; acylbenzoxazines as disclosed in U.S. Pat. No. 6,124,278, and PCT Int'l Pub. No. WO 9951240; carbonylbenzoxazine compounds as disclosed in PCT Int'l Pub. No. WO 03045315; substituted 2,3-benzodiazepin-4-ones as disclosed in U.S. Pat. No. 5,891,871; and benzofurazan compounds as disclosed in U.S. Pat. Nos. 6,110,935; 6,313,115; and PCT Int'l Pub. No. WO 9835950. Examples of benzofurazan compounds include 1-(benzofurazan-5-ylcarbonyl)-4,4-difluoropiperidine and 4-(benzofurazan-5-ylcarbonyl)morpholine. Substituted 5-oxo-5,6,7,8-tetrahydro-4H-1-benzopyrans and benzothiopyrans and related compounds as disclosed in PCT Int'l Pub. No. WO 0075123 are suitable for use as AMPA receptor modulators.

Additional AMPA receptor modulators suitable for use with the practice of the embodiments are amidophosphate derivatives as disclosed in U.S. Pat. No. 6,521,605, and PCT Int'l Pub. No. WO 0006176; monofluoralkyl derivatives as disclosed in PCT Int'l Pub. No. WO 0066546; and substituted quinazolines and analogs thereof as disclosed in PCT Int'l Pub. No. WO 9944612 and quainoxaline compounds and derivatives as disclosed in PCT Int'l Pub. No. WO 07060144. Additional compounds suitable for use as AMPA receptor modulators with practice of the embodiments include 2-ethoxy-4′-[3-(propane-2-sulfonylamino)-thiophen-2-yl]-biphenyl-4-carboxylic acid and derivatives thereof as disclosed in U.S. Pat. Pub. No. 20060276532; pyrrole and pyrazole compounds and derivatives thereof as disclosed in U.S. Pat. Pub. No. 20070066573; and the thiadiazine compounds and derivatives as disclosed in U.S. Pat. Pub. No. 20070004709; and the benzoxazepine compounds and derivatives as disclosed in U.S. Pat. Pub. No. 20040171605. Other compounds suitable for use as AMPA receptor modulators are disclosed in PCT Int'l Pub. Nos. WO 9942456, WO 0006156, WO 0157045, and U.S. Pat. No. 6,617,351.

1. Aniracetam and Ampakines

Ampakines are drugs developed using aniracetam as a structural antecedent. Aniracetam was found to have nootropic effects in whole animals, attributed to its anti-deactivation/anti-desensitization activities at the AMPA receptor (Cumin, R. et al. (1982) Psychopharmacology. (Berl). 78(2):104-11; Ozawa, S. et al. (1991) Neurosci. Res. 12(1):72-82). Ampakines bind to AMPA receptors and prevent or reverse AMPA receptor deactivation and desensitization (Arai, A. C. and Kessler, M. (2007) Curr. Drug Targets 8(5):58-602). These drugs potentiate AMPA-type ionotropic glutamate receptor currents. Proposed clinical applications for ampakines include antipsychotics, cognitive enhancers (PCT Int'l Pub. No. WO 9707799), antidepressives (PCT Int'l Pub. No. WO 9739750), and sexual dysfunction (PCT Int'l Pub. No. WO 9726884). However, knowledge concerning their potential role in affecting motor behavior in the brainstem and spinal cord is limited. Potentially two ampakine formulations, CX546 and CX717, reverse respiratory depression due to opiate intoxication (Funk, G. D. and Greer, J. J. (2006) Am. J. Respir. Crit. Care Med. 174:1384-91; Ren J., et al. (2009) Anesthesiology. 110(6):1364-70), while CX546, may ameliorate the pathological respiratory symptoms of Rett Syndrome (Ogier, M., et al. (2007) J Neurosci, 27:10912-17). These conditions, however, are thought affect the central pattern generator for breathing, i.e., the preBötC, rather than reducing drive to or excitability of motoneurons as is the case for, for example, in OSA or SCI, where central pattern generation generally is unaffected.

2. Benzothiadiazides

A different class of drugs, the benzothiadiazides, also has some of the same pharmacological properties at AMPA receptors. Benzothiadiazide diuretics were the first drugs used for single-agent management of mild to moderate hypertension. Along with β-blockers and angiotensin-converting enzyme inhibitors, these diuretics served as a primary treatment option for hypertension (Gengo F. M., and Gabos C. (1988) Am Heart J 116:305-10). Benzothiadiazides also reverse AMPA receptor desensitization though allosteric modulation (Partin, K. M. et al. (1992) Neuron 11(6):1069-82; Bertolino, M. et al. (1993) Receptors Channels. 1(4):267-78). This, however, is not the only function of these agents in the nervous system. For example, cyclothiazide also inhibits γ-aminobutyric acid Type A (GABA_{A}) and glycine receptors, thus reducing inhibition (Deng, L. and Chen G. (2003) PNAS 100:13025-29; Zhang, X. B., et al. (2007) Mol Pharmacol 73:1195-1202). Additionally, these drugs modulate presynaptic release at fast excitatory synapses and inhibitory synapses (Qi, J., et al. (2006) J Physiol 571:605-18). Non-limiting examples of benzothiadiazide compounds suitable for use in the practice of the embodiments described here are disclosed in PCT Int'l Pub. No. WO 9812185 and PCT Int'l Pub. No. WO 9942456.

There is presently limited understanding of a role for AMPA receptor modulators of deactivation and desensitization in neural transmission. As such, a role for AMPA receptor modulators for treating motoneuronal dysfunctions or disorders associated with weakened neural transmission, such as obstructive sleep apnea, snoring, multiple sclerosis, and spinal cord injury, is currently lacking.

Each of the compounds and compound classes disclosed in the above references may be suitable for use with the embodiments as an AMPA receptor modulator, which can have a plurality of pharmacological properties and mechanisms of action. Each of the compounds and compound classes disclosed in the above references is incorporated by reference in the entirety.

### II. Definitions

It is to be understood that both the foregoing and the following descriptions are examples and explanatory only and are not restrictive of the invention as claimed. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the term “genioglossus muscle” is interchangeable with the term “genioglossus” and the term “benzothiadiazide compound” is interchangeable with “benzothiadiazide.” The term “motoneuronal disorder” and “motoneuronal dysfunction” are interchangeable with the term “motor disorder” and “motoneuronal dysfunction.”

All documents, or portions of documents, cited in this application, including but not limited to patents, patent applications, articles, books, and treatises, are hereby expressly incorporated by reference in their entirety for any purpose.

The term “administration” or “administering” includes routes of introducing an AMPA receptor modulator to a subject to perform its intended function. Examples of routes of administration that may be used include injection (subcutaneous, intravenous, parenterally, intraperitoneally, intrathecal), or oral routes. The pharmaceutical preparations may be given by forms suitable for each administration route. For example, these preparations are administered in tablets or capsule form, by injection, inhalation, eye lotion, eye drops, ointment, suppository, etc. administration by injection, infusion or inhalation; topical by lotion or ointment; and rectal by suppositories. Oral administration is preferred. The injection can be bolus or can be continuous infusion. Depending on the route of administration, an AMPA receptor modulator can be coated with or disposed in a selected material to protect it from natural conditions that may detrimentally affect its ability to perform its intended function. An AMPA receptor modulator can be administered alone, or in conjunction with either another agent or agents as described above or with a pharmaceutically-acceptable carrier, or both. An AMPA receptor modulator can be administered prior to the administration of the other agent, simultaneously with the agent, or after the administration of the agent. Furthermore, an AMPA receptor modulator can also be administered in a proform, which is converted into its active metabolite, or more active metabolite in vivo.

Administration “in combination with” one or more further therapeutic agents includes simultaneous (concurrent) and consecutive administration in any order.

As will be readily apparent to one skilled in the art, the useful in vivo dosage to be administered and the particular mode of administration will vary depending upon the age, weight and mammalian species treated, the particular compounds employed, and the specific use for which these compounds are employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved.

As used herein, an “increase” or “decrease” in a measurement, unless otherwise specified, is typically in comparison to a baseline value. For example, an increase in genioglossus activity or tone may be in comparison to a baseline value of such measurements before or without administration of an AMPA receptor modulator. In some instances an increase or decrease in a measurement can be evaluated based on the context in which the term is used. For example, an increase or decrease in a measurement can be evaluated based on comparison to control or placebo.

As used herein, the terms “amplify,” “amplification,” “potentiate,” or “potentiation” are interchangeable with the term “increase” as used herein, and can refer to the activity of the genioglossus, hypoglossal XII nerve or neurons, motoneurons, or neurons generally.

The terms “output,” “discharge,” “activity,” “command,” and “drive” as used herein are interchangeable terms that can refer, inter alia, to a parameter, function, or physiological action of the genioglossus, hypoglossal XII nerve or neurons, motoneurons, or neurons generally. A person of ordinary skill in the art will understand that output, discharge, activity, command, and drive can be measured and represented by units of measurement. For example, hypoglossal XII nerve activity, output, discharge, command, or drive can be represented and used interchangeably with integrated XII amplitude (∫XII). As another non-limiting example, genioglossus activity, output, or discharge can be represented by and used interchangeably with integrated GG amplitude (∫GG).

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN, polyethylene glycol (PEG).

The term “effective amount” includes an amount effective, at dosages and for periods of time necessary, to achieve the desired result, e.g., sufficient to increase genioglossus activity or tone, or sufficient to treat or prevent a motoneuronal dysfunction or disorder including, for example, obstructive sleep apnea and snoring, in a patient or subject. An effective amount of an AMPA receptor modulator may vary according to factors such as the disease state, age, and weight of the subject, and the ability of an AMPA receptor modulator to elicit a desired response in the subject. Dosage regimens may be adjusted to provide the optimum therapeutic response. An effective amount is also one in which any toxic or detrimental effects (e.g., side effects) of an AMPA receptor modulator are outweighed by the therapeutically beneficial effects.

“Ameliorate,” “amelioration,” “improve,” “improvement” or the like refers to, for example, a detectable improvement or a detectable change consistent with improvement that occurs in a subject or in at least a minority of subjects, e.g., in at least about 2%, 5%, 10%, 15%, 20%, 25%, 30%, 40%, 50%, 60%, 70%, 75%, 80%, 85%, 90%, 95%, 98%, 100% or in a range between about any two of these values. Such improvement or change may be observed in treated subjects as compared to subjects not treated with an AMPA receptor modulator, where the untreated subjects have, or are subject to developing, the same or similar disease, condition, symptom or the like. Amelioration of a disease, condition, symptom or assay parameter may be determined subjectively or objectively, e.g., self assessment by a subject(s), by a clinician's assessment or by conducting an appropriate assay or measurement, including, e.g., a quality of life assessment, a slowed progression of a disease(s) or condition(s), a reduced severity of a disease(s) or condition(s), or a suitable assay(s) for the level or activity(ies) of a biomolecule(s), cell(s) or by detection of a motoneuronal dysfunction or disorder including for example, obstructive sleep apnea and snoring, in a subject. Amelioration may be transient, prolonged or permanent or it may be variable at relevant times during or after an AMPA receptor modulator is administered to a subject or is used in an assay or other method described herein or a cited reference.

The “modulation” of, e.g., a symptom, level or biological activity of a molecule, or the like, refers, for example, that the symptom or activity, or the like is detectably increased or decreased. Such increase or decrease may be observed in treated subjects as compared to subjects not treated with an AMPA receptor modulator, where the untreated subjects have, or are subject to developing, the same or similar disease, condition, symptom or the like. Such increases or decreases may be at least about 2%, 5%, 10%, 15%, 20%, 25%, 30%, 40%, 50%, 60%, 70%, 75%, 80%, 85%, 90%, 95%, 98%, 100%, 150%, 200%, 250%, 300%, 400%, 500%, 1000% or more or within any range between any two of these values. Modulation may be determined subjectively or objectively, e.g., by the subject's self assessment, by a clinician's assessment or by conducting an appropriate assay or measurement, including, e.g., quality of life assessments or suitable assays for the level or activity of molecules, for example receptors. Modulation may be transient, prolonged or permanent or it may be variable at relevant times during or after an AMPA receptor modulator is administered to a subject or is used in an assay or other method described herein or a cited reference, e.g., within times described herein.

The term “obtaining” as in “obtaining an AMPA receptor modulator” is intended to include purchasing, synthesizing or otherwise acquiring an AMPA receptor modulator.

The phrases “parenteral administration” and “administered parenterally” as used herein includes, for example, modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The language “a prophylactically effective amount” of a compound refers to an amount of an AMPA receptor modulator which is effective, upon single or multiple dose administration to the subject, in preventing or treating a motoneuronal dysfunction or disorder including, for example, obstructive sleep apnea and snoring.

The term “pharmaceutical agent composition” (or agent or drug) as used herein refers to a chemical compound, composition, agent or drug capable of inducing a desired therapeutic effect when properly administered to a patient. It does not necessarily require more than one type of ingredient.

The compositions may be in the “pharmaceutical form” of tablets, capsules, powders, granules, lozenges, liquid or gel preparations. Tablets and capsules for oral administration may be in a form suitable for unit dose presentation and may contain conventional excipients. Examples of these are: binding agents such as syrup, acacia, gelatin, sorbitol, tragacanth, and polyvinylpyrrolidone; fillers such as lactose, sugar, maize-starch, calcium phosphate, sorbitol or glycine; tableting lubricants, such as magnesium stearate, silicon dioxide, talc, polyethylene glycol or silica; disintegrants, such as potato starch; or acceptable wetting agents, such as sodium lauryl sulfate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, e.g., sorbitol, syrup, methyl cellulose, glucose syrup, gelatin, hydrogenated edible fats, emulsifying agents, e.g., lecithin, sorbitan monooleate, or acacia; non-aqueous vehicles (including edible oils), e.g., almond oil, fractionated coconut oil, oily esters such as glycerine, propylene glycol, or ethyl alcohol; preservatives such as methyl or propyl p-hydroxybenzoate or sorbic acid, and, if desired, conventional flavoring or coloring agents.

The term “pharmaceutical preparation” or “pharmaceutical formulation” refers to a pharmaceutical agent composition that can be in a pharmaceutical form described herein.

The phrases “systemic administration,” “administered systemically,” “peripheral administration,” and “administered peripherally,” as used herein mean the administration of an AMPA receptor modulator, drug or other material, such that it enters the subject's system and, thus, is subject to metabolism and other like processes, for example, subcutaneous administration.

The language “therapeutically effective amount” of an AMPA receptor modulator refers to an amount of an AMPA receptor modulator which is effective, upon single or multiple dose administration to the subject, in increasing genioglossus muscle tone or activity in a subject. “Therapeutically effective amount” also refers to the amount of a therapy (e.g., a composition comprising an AMPA receptor modulator), which is sufficient to reduce or inhibit a motoneuronal dysfunction or disorder including, for example, obstructive sleep apnea and snoring, in a subject.

As used herein, the terms “prevent,” “preventing,” and “prevention” refer to the prevention of the recurrence, onset, or development of a motoneuronal dysfunction or disorder including, for example, obstructive sleep apnea and snoring.

As used herein, the term “inhibit” refers to “prevention” or reduction.

As used herein, the term “prophylactically effective amount” refers to the amount of a therapy (e.g., a composition comprising an AMPA receptor modulator) which is sufficient to result in the prevention of the development, recurrence, or onset of a motoneuronal dysfunction or disorder including, for example, obstructive sleep apnea and snoring, or to enhance or improve the prophylactic effect(s) of another therapy.

As used herein, “subject” includes organisms which are capable of suffering from a motoneuronal dysfunction or disorder including, for example, obstructive sleep apnea and snoring, treatable by an AMPA receptor modulator or who could otherwise benefit from the administration of an AMPA receptor modulator as described herein, such as human and non-human animals. Preferred human animals include human subjects. The term “non-human animals” of the invention includes all vertebrates, e.g., mammals, e.g., rodents, e.g., mice, rats, and non-mammals, such as non-human primates, e.g., sheep, dog, cow, chickens, amphibians, reptiles, etc.

### III. Pharmaceutical Forms, Methods, and Kits

Several embodiments described herein relate to the discovery of AMPA receptor modulators that increase genioglossus muscle activity. Various embodiments relate to the use of AMPA receptor modulators to reduce or inhibit a motor dysfunctions or disorders such as obstructive sleep apnea, snoring, multiple sclerosis, spinal cord injury, e.g., crush, partial or complete transection, motor neuron diseases, motor weakness due to aging, stroke, tumor, hemorrhage, degenerative or wasting diseases, and spasticity.

In one embodiment, AMPA receptor modulators are identified or screened using an in vitro brain slice assay. For example, a thin brain slice preparation is taken from an anesthetized neonatal rat 0 to 4 days of age and incubated in cerebrospinal fluid. A brain slice preparation from the medulla produces endogenous inspiratory activity that can be measured from a suction electrode attached to the hypoglossal (XII) nerve rootlets. AMPA receptor modulators can be identified as producing an effect on hypoglossal motor activity when this activity is compared between periods before and after applying the compound being studied. In one aspect, AMPA receptor modulators that increase hypoglossal motor activity are identified or screened.

In one embodiment, the AMPA receptor modulator is an ampakine. In one aspect of the embodiment, the ampakine is CX546. In other embodiments, the ampakine is aniracetam and its derivatives, benzoyl piperidines, pyrrolidines, or benzoxazines.

In one embodiment, the AMPA receptor modulator is a benzothiadiazide. In one aspect of the embodiment, the benzothiadiazide is cyclothiazide. In other aspects of the embodiment, the benzothiadiazide is hydrochlorothiazide, chlorothiazide, hydroflumethiazide, tricholoromethiazide, althiazide, IDRA-21, S18986, diazoxide, or any of their derivatives.

In various embodiments, AMPA receptor modulators separately, together, or in combination with agonists, antagonists, or modulators (allosteric and non-allosteric) of receptors of, reuptake transporters of, or enzymes related to synthesis or metabolism of serotonin, norepinephrine, epinephrine, dopamine, γ-aminobutyric acid (GABA), glycine, acetylcholine, cannabinoid, adenosine, adenosine, guanosine, or uridine triphosphate (ATP, GTP, UTP) or their metabolites, or brain-derived neurotrophic factor (BDNF) or its derivatives, are used for increasing genioglossus muscle tone or activity.

In various embodiments, AMPA receptor modulators separately, together, or in combination with agonists, antagonists, or modulators (allosteric and non-allosteric) of receptors of, reuptake transporters of, or enzymes related to synthesis or metabolism of serotonin, norepinephrine, epinephrine, dopamine, γ-aminobutyric acid (GABA), glycine, acetylcholine, cannabinoid, adenosine, adenosine, guanosine, or uridine triphosphate (ATP, GTP, UTP) or their metabolites, or brain-derived neurotrophic factor (BDNF) or its derivatives, are used for the treatment of motoneuronal dysfunctions or disorders including, for example, obstructive sleep apnea, snoring, multiple sclerosis, and spinal cord injury.

In one embodiment, a method of increasing genioglossus muscle tone in a subject is provided. The subject is administered an effective amount of an AMPA receptor modulator sufficient to increase genioglossus muscle activity. In one aspect, the AMPA receptor modulator is an ampakine. The ampakine in one aspect of the embodiment is CX546. In other aspects, the ampakine is a compound referenced herein. In yet another aspect, the AMPA receptor modulator is a benzothiadiazide. The benzothiadiazide in one aspect is cyclothiazide. The benzothiadiazide in other aspects are hydrochlorothiazide, chlorothiazide, hydroflumethiazide, tricholoromethiazide, althiazide, IDRA-21, S18986, diazoxide, or any of their derivatives. In other aspects, the benzothiadiazide is referenced herein.

In one embodiment, genioglossus activity is measured by electromyogram (EMG).

In one embodiment, the increase of genioglossus activity induced by administration of an AMPA receptor modulator has a duration greater than about 0.25, 0.5, 0.75, 1, greater than about 2, 3, 4, 5, 6, 7, 8, 9, 10, 11, 12, 13, 14, 15, 16, 17, 18, 19, 20, 21, 22, 23, 24, greater than about 48, 72, greater than 72 hours, or in a range between about any two of these values. In one aspect of the embodiment, administration of an AMPA receptor modulator induces the increase of genioglossus activity for a duration greater than about 0.25, 0.5, 0.75, 1, greater than about 2, 3, 4, 5, 6, 7, 8, 9, 10, 11, 12, 13, 14, 15, 16, 17, 18, 19, 20, 21, 22, 23, 24, greater than about 48, 72, greater than 72 hours, or in a range between about any two of these values without significantly affecting respiratory rate, tidal volume, or heart rate.

In one embodiment, the increase of genioglossus activity induced by administration of CX546 has a duration greater than about 2 hours.

In one embodiment, the increase of genioglossus activity induced by administration of cyclothiazide has a duration greater than about 2 hours.

In one embodiment, the increase of genioglossus activity induced by administration of an AMPA receptor modulator is greater than about 5%, 10%, 15%, 20%, 25%, 30%, 35%, 40%, 45%, 50%, 55%, 60%, 65%, 70%, 75%, 80%, 90%, 100%, 105%, 110%, 115%, 120%, 125%, 130%, 135%, 140%, 145%, 150%, 155%, 160%, 165%, 170%, 175%, 180%, 190%, 200%, 205%, 210%, 215%, 220%, 225%, 230%, 235%, 240%, 245%, 250%, 255%, 260%, 265%, 270%, 275%, 280%, 290%, 300%, 325%, 350%, 375%, 400%, 425%, 450%, 475%, 500%, greater than about 1000%, 2000%, 3000%, 4000%, 5000%, greater than 5000%, or in a range between about any two of these values for any duration or at any time point compared to pre-administration, control, or placebo.

In one embodiment, the increase of genioglossus activity induced by administration of CX546 is greater than about 15%.

In one embodiment, the increase of genioglossus activity induced by administration of cyclothiazide is greater than about 50%. In one embodiment, the increase of genioglossus activity induced by administration of cyclothiazide is greater than about 150%. In one embodiment, the increase of genioglossus activity induced by administration of cyclothiazide is greater than about 300%. In one embodiment, the increase of genioglossus activity induced by administration of cyclothiazide is greater than about 600%.

In one embodiment, the effective amount of the ampakine sufficient to increase genioglossus activity is a dosage administered to a subject of about 1 μg/kg, 50 μg/kg, 100 μg/kg, 150 μg/kg, 200 μg/kg, 250 μg/kg, 300 μg/kg, 350 μg/kg, 400 μg/kg, 500 μg/kg, 550 μg/kg, 600 μg/kg, 700 μg/kg, 800 μg/kg, 900 μg/kg, 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 75 mg/kg, 100 mg/kg, greater than 100 mg/kg (mass of ampakine/mass of subject), or in a range between about any two of these values. In another embodiment, the effective amount of the ampakine can be administered as a fixed dosage irrespective of the subject's mass. For example, the effective amount of the ampakine sufficient to increase genioglossus activity is a fixed dose of about 1 μg, 50 μg, 75 μg, 100 μg, 150 μg, 200 μg, 250 μg, 300 μg, 350 μg, 400 μg, 500 μg, 550 μg, 600 μg, 700 μg, 800 μg, 900 μg, 1 mg, 5 mg, 10 mg, 15 mg, 20 mg, 25 mg, 30 mg, 35 mg, 40 mg, 45 mg, 50 mg, 75 mg, 100 mg, 200 mg, 300 mg, 400 mg, 500 mg, 600 mg, 700 mg, 800 mg, 900 mg, 1000 mg, 1250 mg, 1500 mg, 1750 mg, 2000 mg, 2250 mg, 2500 mg, 2750 mg, 3000 mg, 3500 mg, 4000 mg, 4500 mg, 5000 mg greater than 5000 mg, or in a range between about any two of these values. In one aspect, the ampakine is CX546.

In one embodiment, the effective amount of the benzothiadiazide sufficient to increase genioglossus activity is a dosage administered to a subject of about 1 μg/kg, 50 μg/kg, 100 μg/kg, 150 μg/kg, 200 μg/kg, 250 μg/kg, 300 μg/kg, 350 μg/kg, 400 μg/kg, 500 μg/kg, 550 μg/kg, 600 μg/kg, 700 μg/kg, 800 μg/kg, 900 μg/kg, 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 75 mg/kg, 100 mg/kg, greater than 100 mg/kg (mass of benzothiadiazide/mass of subject), or in a range between about any two of these values. In another embodiment, the effective amount of the benzothiadiazide can be administered as a fixed dosage irrespective of the subject's mass. For example, the effective amount of the benzothiadiazide sufficient to increase genioglossus activity is a fixed dose of about 1 μg, 50 μg, 75 μg, 100 μg, 150 μg, 200 μg, 250 μg, 300 μg, 350 μg, 400 μg, 500 μg, 550 μg, 600 μg, 700 μg, 800 μg, 900 μg, 1 mg, 5 mg, 10 mg, 15 mg, 20 mg, 25 mg, 30 mg, 35 mg, 40 mg, 45 mg, 50 mg, 75 mg, 100 mg, 200 mg, 300 mg, 400 mg, 500 mg, 600 mg, 700 mg, 800 mg, 900 mg, 1000 mg, 1250 mg, 1500 mg, 1750 mg, 2000 mg, 2250 mg, 2500 mg, 2750 mg, 3000 mg, 3500 mg, 4000 mg, 4500 mg, 5000 mg, greater than 5000 mg, or in a range between about any two of these values. In one aspect, the benzothiadiazide is cyclothiazide.

In various embodiments, the effective amount of an AMPA receptor modulator sufficient to increase genioglossus muscle activity is also sufficient to reduce or inhibit snoring. For example, administration of any dosage or fixed dose of an ampakine or benzothiadiazide, described herein, can be sufficient to increase genioglossus muscle activity by any percent, described herein, and reduce or inhibit snoring in a subject.

In various embodiments, the effective amount of an AMPA receptor modulator sufficient to increase genioglossus muscle activity is also sufficient to reduce or inhibit obstructive sleep apnea. For example, administration of any dosage or fixed dose of an ampakine or benzothiadiazide, described herein, can be sufficient to increase genioglossus muscle activity by any percent, described herein, and reduce or inhibit obstructive sleep apnea in a subject.

In one embodiment, a method of treating snoring in a subject is provided which includes administering an effective amount of an AMPA receptor modulator sufficient to reduce or inhibit snoring. The effective amount of the AMPA receptor modulator sufficient to reduce or inhibit snoring can be administered in a dosage of about 1 μg/kg, 50 μg/kg, 100 μg/kg, 150 μg/kg, 200 μg/kg, 250 μg/kg, 300 μg/kg, 350 μg/kg, 400 μg/kg, 500 μg/kg, 550 μg/kg, 600 μg/kg, 700 μg/kg, 800 μg/kg, 900 μg/kg, 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 75 mg/kg, or 100 mg/kg (mass of AMPA receptor modulator/mass of subject). In another embodiment, the effective amount of the AMPA receptor modulator can be administered as a fixed dosage irrespective of the subject's mass. For example, the effective amount of the AMPA receptor modulator sufficient to reduce or inhibit snoring is a fixed dose of about 1 μg, 50 μg, 75 μg, 100 μg, 150 μg, 200 μg, 250 μg, 300 μg, 350 μg, 400 μg, 500 μg, 550 μg, 600 μg, 700 μg, 800 μg, 900 μg, 1 mg, 5 mg, 10 mg, 15 mg, 20 mg, 25 mg, 30 mg, 35 mg, 40 mg, 45 mg, 50 mg, 75 mg, 100 mg, 200 mg, 300 mg, 400 mg, 500 mg, 600 mg, 700 g, 800 mg, 900 mg, 1000 mg, 1250 mg, 1500 mg, 1750 mg, 2000 mg, 2250 mg, 2500 mg, 2750 mg, 3000 mg, 3500 mg, 4000 mg, 4500 mg, or 5000 mg.

In one embodiment, a method of treating snoring in a subject is provided which includes administering an effective amount of an ampakine sufficient to reduce or inhibit snoring. The effective amount of an ampakine sufficient to reduce or inhibit snoring can be administered in a dosage of about 1 μg/kg, 50 μg/kg, 100 μg/kg, 150 μg/kg, 200 μg/kg, 250 μg/kg, 300 μg/kg, 350 μg/kg, 400 μg/kg, 500 μg/kg, 550 μg/kg, 600 μg/kg, 700 μg/kg, 800 μg/kg, 900 μg/kg, 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 75 mg/kg, or 100 mg/kg (mass of ampakine/mass of subject). In another embodiment, the effective amount of the ampakine can be administered as a fixed dosage irrespective of the subject's mass. For example, the effective amount of the ampakine sufficient to reduce or inhibit snoring is a fixed dose of about 1 μg, 50 μg, 75 μg, 100 μg, 150 μg, 200 μg, 250 μg, 300 μg, 350 μg, 400 μg, 500 μg, 550 μg, 600 μg, 700 μg, 800 μg, 900 μg, 1 mg, 5 mg, 10 mg, 15 mg, 20 mg, 25 mg, 30 mg, 35 mg, 40 mg, 45 mg, 50 mg, 75 mg, 100 mg, 200 mg, 300 mg, 400 mg, 500 mg, 600 mg, 700 mg, 800 mg, 900 mg, 1000 mg, 1250 mg, 1500 mg, 1750 mg, 2000 mg, 2250 mg, 2500 mg, 2750 mg, 3000 mg, 3500 mg, 4000 mg, 4500 mg, or 5000 mg. In one aspect, the ampakine is CX546.

In one embodiment, a method of treating snoring in a subject is provided which includes administering an effective amount of a benzothiadiazide compound sufficient to reduce or inhibit snoring. The effective amount of a benzothiadiazide compound sufficient to reduce or inhibit snoring can be administered in a dosage of about 1 μg/kg, 50 μg/kg, 100 μg/kg, 150 μg/kg, 200 μg/kg, 250 μg/kg, 300 μg/kg, 350 μg/kg, 400 μg/kg, 500 μg/kg, 550 μg/kg, 600 μg/kg, 700 μg/kg, 800 μg/kg, 900 μg/kg, 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 75 mg/kg, or 100 mg/kg (mass of benzothiadiazide/mass of subject). In another embodiment, the effective amount of the benzothiadiazide compound can be administered as a fixed dosage irrespective of the subject's mass. For example, the effective amount of the benzothiadiazide compound sufficient to reduce or inhibit snoring is a fixed dose of about 1 μg, 50 μg, 75 μg, 100 μg, 150 μg, 200 μg, 250 μg, 300 μg, 350 μg, 400 μg, 500 μg, 550 μg, 600 μg, 700 μg, 800 μg, 900 μg, 1 mg, 5 mg, 10 mg, 15 mg, 20 mg, 25 mg, 30 mg, 35 mg, 40 mg, 45 mg, 50 mg, 75 mg, 100 mg, 200 mg, 300 mg, 400 mg, 500 mg, 600 mg, 700 mg, 800 mg, 900 mg, 1000 mg, 1250 mg, 1500 mg, 1750 mg, 2000 mg, 2250 mg, 2500 mg, 2750 mg, 3000 mg, 3500 mg, 4000 mg, 4500 mg, or 5000 mg. In one aspect, the benzothiadiazide compound is cyclothiazide.

In one embodiment, a method of treating obstructive sleep apnea in a subject is provided which includes administering an effective amount of a benzothiadiazide compound sufficient to reduce or inhibit obstructive sleep apnea. The effective amount of a benzothiadiazide compound sufficient to reduce or inhibit obstructive sleep apnea can be administered in a dosage of about 1 μg/kg, 50 μg/kg, 100 μg/kg, 150 μg/kg, 200 μg/kg, 250 μg/kg, 300 μg/kg, 350 μg/kg, 400 μg/kg, 500 μg/kg, 550 μg/kg, 600 μg/kg, 700 μg/kg, 800 μg/kg, 900 μg/kg, 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 75 mg/kg, or 100 mg/kg (mass of benzothiadiazide/mass of subject). In another embodiment, the effective amount of the benzothiadiazide compound can be administered as a fixed dosage irrespective of the subject's mass. For example, the effective amount of the benzothiadiazide compound sufficient to reduce or inhibit obstructive sleep apnea is a fixed dose of about 1 μg, 50 μg, 75 μg, 100 μg, 150 μg, 200 μg, 250 μg, 300 μg, 350 μg, 400 μg, 500 μg, 550 μg, 600 μg, 700 μg, 800 μg, 900 μg, 1 mg, 5 mg, 10 mg, 15 mg, 20 mg, 25 mg, 30 mg, 35 mg, 40 mg, 45 mg, 50 mg, 75 mg, 100 mg, 200 mg, 300 mg, 400 mg, 500 mg, 600 mg, 700 mg, 800 mg, 900 mg, 1000 mg, 1250 mg, 1500 mg, 1750 mg, 2000 mg, 2250 mg, 2500 mg, 2750 mg, 3000 mg, 3500 mg, 4000 mg, 4500 mg, or 5000 mg.

In one embodiment, a method of treating obstructive sleep apnea in a subject is provided which includes administering an effective amount of cyclothiazide sufficient to reduce or inhibit obstructive sleep apnea. The effective amount of cyclothiazide sufficient to reduce or inhibit obstructive sleep apnea can be administered in a dosage of about 1 μg/kg, 50 μg/kg, 100 μg/kg, 150 μg/kg, 200 μg/kg, 250 μg/kg, 300 μg/kg, 350 μg/kg, 400 μg/kg, 500 μg/kg, 550 μg/kg, 600 μg/kg, 700 μg/kg, 800 μg/kg, 900 μg/kg, 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 75 mg/kg, or 100 mg/kg (mass of cyclothiazide/mass of subject). In another embodiment, the effective amount of cyclothiazide can be administered as a fixed dosage irrespective of the subject's mass. For example, the effective amount of cyclothiazide sufficient to reduce or inhibit obstructive sleep apnea is a fixed dose of about 1 μg, 50 μg, 75 μg, 100 μg, 150 μg, 200 μg, 250 μg, 300 μg, 350 μg, 400 μg, 500 μg, 550 μg, 600 μg, 700 μg, 800 μg, 900 μg, 1 mg, 5 mg, 10 mg, 15 mg, 20 mg, 25 mg, 30 mg, 35 mg, 40 mg, 45 mg, 50 mg, 75 mg, 100 mg, 200 mg, 300 mg, 400 mg, 500 mg, 600 mg, 700 mg, 800 mg, 900 mg, 1000 mg, 1250 mg, 1500 mg, 1750 mg, 2000 mg, 2250 mg, 2500 mg, 2750 mg, 3000 mg, 3500 mg, 4000 mg, 4500 mg, or 5000 mg.

In one embodiment, a method of treating multiple sclerosis (MS) in a subject is provided which includes administering an effective amount of an AMPA receptor modulator sufficient to reduce or inhibit motor or cognitive deficiency in the subject resulting from multiple sclerosis. In one aspect, the motor deficiency in the subject resulting from multiple sclerosis can be muscle weakness or ataxia associated with MS outbreaks. The effective amount of the AMPA receptor modulator sufficient to reduce or inhibit motor or cognitive deficiency resulting from MS can be administered in a dosage of about 1 μg/kg, 50 μg/kg, 100 μg/kg, 150 μg/kg, 200 μg/kg, 250 μg/kg, 300 μg/kg, 350 μg/kg, 400 μg/kg, 500 μg/kg, 550 μg/kg, 600 μg/kg, 700 μg/kg, 800 μg/kg, 900 μg/kg, 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 75 mg/kg, or 100 mg/kg (mass of AMPA receptor modulator/mass of subject). In another embodiment, the effective amount of the AMPA receptor modulator can be administered as a fixed dosage irrespective of the subject's mass. For example, the effective amount of the AMPA receptor modulator sufficient to reduce or inhibit motor or cognitive deficiency resulting from MS is a fixed dose of about 1 μg, 50 μg, 75 μg, 100 μg, 150 μg, 200 μg, 250 μg, 300 μg, 350 μg, 400 μg, 500 μg, 550 μg, 600 μg, 700 μg, 800 μg, 900 μg, 1 mg, 5 mg, 10 mg, 15 mg, 20 mg, 25 mg, 30 mg, 35 mg, 40 mg, 45 mg, 50 mg, 75 mg, 100 mg, 200 mg, 300 mg, 400 mg, 500 mg, 600 mg, 700 mg, 800 mg, 900 mg, 1000 mg, 1250 mg, 1500 mg, 1750 mg, 2000 mg, 2250 mg, 2500 mg, 2750 mg, 3000 mg, 3500 mg, 4000 mg, 4500 mg, or 5000 mg. In one aspect, the AMPA receptor modulator is an ampakine. In the same aspect, the ampakine is CX546. In another aspect, the AMPA receptor modulator is a benzothiadiazide. In the same aspect, the benzothiadiazide is cyclothiazide.

In one embodiment, a method of treating spinal cord injury in a subject is provided which includes administering an effective amount of an AMPA receptor modulator sufficient to improve sensory or motor function in the subject. The effective amount of the AMPA receptor modulator sufficient to improve sensory or motor function in the subject can be administered in a dosage of about 1 μg/kg, 50 μg/kg, 100 μg/kg, 150 μg/kg, 200 μg/kg, 250 μg/kg, 300 μg/kg, 350 μg/kg, 400 μg/kg, 500 μg/kg, 550 μg/kg, 600 μg/kg, 700 μg/kg, 800 μg/kg, 900 μg/kg, 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 75 mg/kg, or 100 mg/kg (mass of AMPA receptor modulator/mass of subject). In another embodiment, the effective amount of the AMPA receptor modulator can be administered as a fixed dosage irrespective of the subject's mass. For example, the effective amount of the AMPA receptor modulator sufficient to improve sensory or motor function in the subject is a fixed dose of about 1 μg, 50 μg, 75 μg, 100 μg, 150 μg, 200 μg, 250 μg, 300 μg, 350 μg, 400 μg, 500 μg, 550 μg, 600 μg, 700 μg, 800 μg, 900 μg, 1 mg, 5 mg, 10 mg, 15 mg, 20 mg, 25 mg, 30 mg, 35 mg, 40 mg, 45 mg, 50 mg, 75 mg, 100 mg, 200 mg, 300 mg, 400 mg, 500 mg, 600 mg, 700 mg, 800 mg, 900 mg, 1000 mg, 1250 mg, 1500 mg, 1750 mg, 2000 mg, 2250 mg, 2500 mg, 2750 mg, 3000 mg, 3500 mg, 4000 mg, 4500 mg, or 5000 mg. In one aspect, the AMPA receptor modulator is an ampakine. In the same aspect, the ampakine is CX546. In another aspect, the AMPA receptor modulator is a benzothiadiazide. In the same aspect, the benzothiadiazide is cyclothiazide.

One embodiment includes articles of manufacture that comprise, for example, a container holding a pharmaceutical composition suitable for oral administration of an AMPA receptor modulator in combination with printed labeling instructions providing a discussion of when a particular dosage form reduces or inhibits a motor dysfunctions or disorders including, for example, obstructive sleep apnea, snoring, multiple sclerosis, and spinal cord injury. The dosage can be modified for administration to a subject suffering from obstructive sleep apnea or snoring, or include labeling for administration to a subject suffering from obstructive sleep apnea, snoring, multiple sclerosis, or spinal cord injury. Examples of dosage forms and administration protocols are described herein. The composition will be contained in any suitable container capable of holding and dispensing the dosage form and which will not significantly interact with the composition and will further be in physical relation with the appropriate labeling. The labeling instructions may be consistent with the methods of treatment as described hereinbefore. The labeling may be associated with the container by any means that maintain a physical proximity of the two, by way of non-limiting example, they may both be contained in a packaging material such as a box or plastic shrink wrap or may be associated with the instructions being bonded to the container such as with glue that does not obscure the labeling instructions or other bonding or holding means.

In one embodiment, the instructions will inform or advise a health care worker, prescribing physician, a pharmacist, or a subject that they should advise a patient suffering from a motor dysfunctions or disorders including, for example, obstructive sleep apnea, snoring, multiple sclerosis, or spinal cord injury, that administration of an AMPA receptor modulator may induce side effects.

Packaged compositions are also provided, and may comprise a therapeutically effective amount of AMPA receptor modulator tablets or capsules. Kits are also provided herein, for example, kits for treating a motoneuronal dysfunction or disorder including, for example, obstructive sleep apnea, snoring, multiple sclerosis, and spinal cord injury, in a subject. The kits may contain, for example, an AMPA receptor modulator and instructions for use when treating a subject for a motor dysfunctions or disorders including, for example, obstructive sleep apnea, snoring, multiple sclerosis, and spinal cord injury. The instructions for use may contain prescribing information, dosage information, storage information, and the like.

It should be appreciated that embodiments of the invention should not be construed to be limited to the examples, which are now described; rather, the invention should be construed to include any and all applications provided herein and all equivalent variations within the skill of the ordinary artisan.

### EXAMPLES

### Example 1

### Medullary Slice Preparations

Experiments were performed on medullary slice preparations from 0-4 day-old neonatal rats that retain functional respiratory networks. Details of the preparation have been previously described. See Funk et al. (1993) J Neurophysiol 70:1497-1515. Briefly, neonatal rats were anesthetized with isoflurane, decerebrated, and the neuraxis isolated by dissection in artificial cerebrospinal fluid (ACSF) [(in mM) 128 NaCl, 3.0 KCl, 1.5 CaCl₂, 1.0 mgSO₄, 21 NaHCO₃, 0.5 Na₂HPO₄, and 30 D-glucose] equilibrated with 95% 0₂-5% CO₂ at 27-28° C. The neuraxis was sectioned with a vibratome in the transverse plane starting from the rostral medulla to within 150 μm nucleus ambiguous (Smith, J. C. et al., (1991) Science 254(5032):726-729. A single, 700 μm thick transverse slice was then cut, transferred to a 1.5 ml recording chamber and held in place by stainless steel harp. The slice was continuously superfused (≧3 ml/min) by ACSF with extracellular K⁺ concentration raised to 9 mM to maintain rhythmic output.

XII motoneurons were selected according to a hierarchy of criteria. Selection criteria for respiratory motoneurons are described by Funk et al. (1993) J Neurophysiol 70:1497-1515. They include a multipolar somata located in the ventral portion of XII nucleus and the presence of excitatory synaptic drive currents coincident with activity in the XII nerve roots.

Respiratory motor output was recorded from cut ends of XII nerve roots rectified, filtered and integrated. Peak amplitude was obtained from signals of XII nerve discharge. The activity in individual XII motoneurons was measured via whole-cell voltage-clamp techniques.

Cyclothiazide, CX546, and aniracetam were introduced by bath application to determine the effects of cyclothiazide, CX546, and aniracetam on respiratory activity. Stable respiratory activity was recorded for at least 30 minutes preceding drug application. After removal of the drug, recordings continued for over 12 hours.

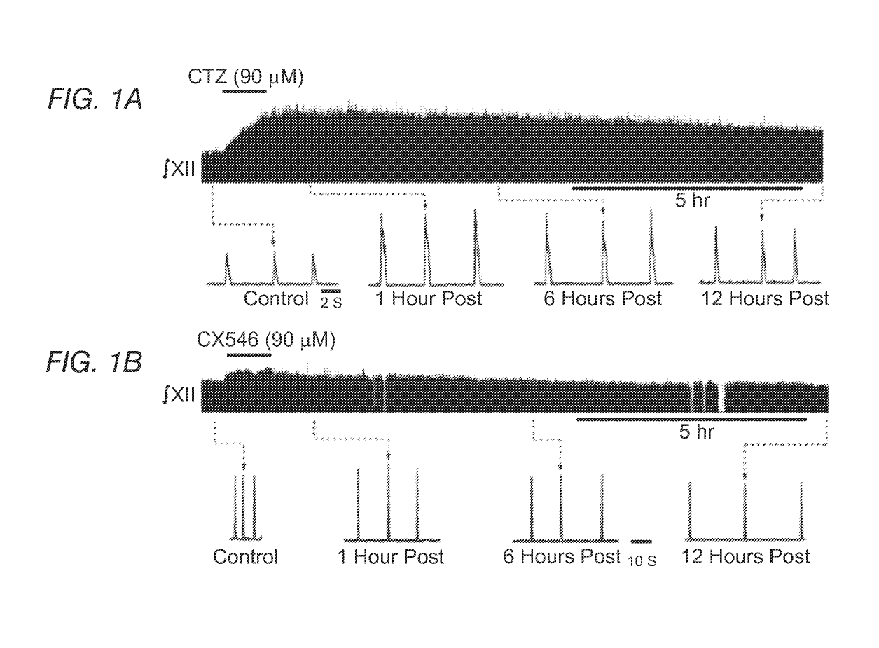

Integrated hypoglossal (∫XII) nerve recordings measured the effects of a 1-hour bath application of 90 μM cyclothiazide (CTZ) (FIG. 1A) or 90 μM CX546 (FIG. 1B) to the in vitro neonatal medullary slice preparation. As shown in FIG. 1A, short-term (1 hour) in vitro application of CTZ to the slice preparation induces a dramatic (2-fold) and persistent (>12 hours) potentiation of hypoglossal (∫XII) nerve discharge amplitude with minor effects on respiratory rate (frequency). This potentiation of discharge amplitude persists long after the drug is removed, while rate effects subside. These data suggest that single drug treatment with a benzothiadiazide can induce long-lasting changes in neuronal function that can rectify defects in neuronal function. As shown in FIG. 1B, short-term (1 hour) in vitro application of CX546 induces an increase in ∫XII nerve discharge and respiration rate that subsides over time with drug removal. Removal of CX546 eliminated some but not all of the potentiation induced by application of the drug, suggesting that an ampakine can be used as a once-nightly, daily, or other recurring schedule drug depending on the motoneuronal condition.

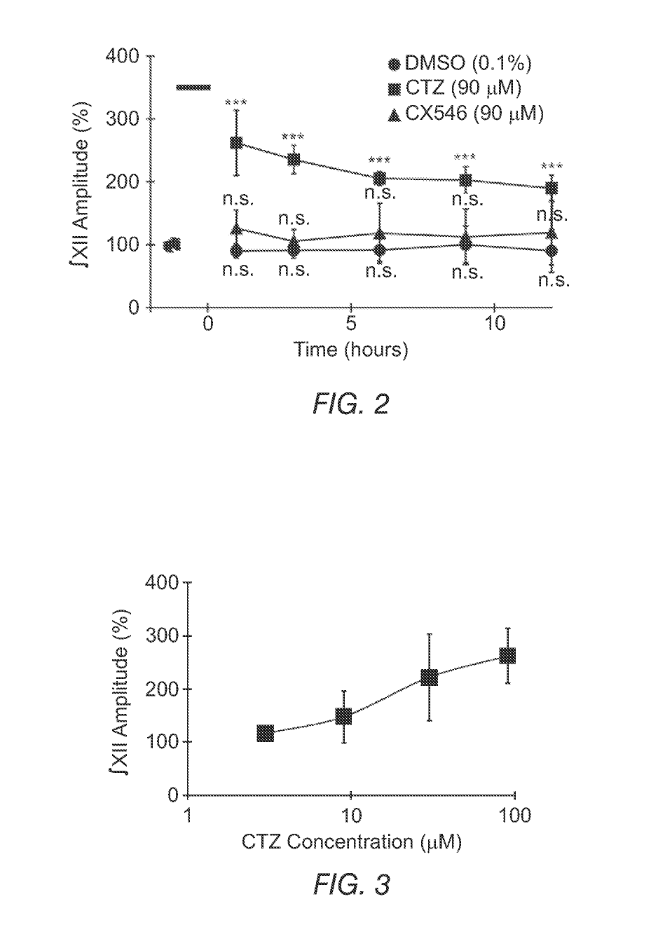

FIG. 2 summarizes group data for the percent increase of ∫XII amplitude following short-duration application of cyclothiazide or CX546. Short-duration application of cyclothiazide but not CX546 causes long-lasting potentiation of ∫XII nerve amplitude. Group data showing normalized ∫XII nerve amplitude for control and 1-12 hours following a 1 hour application (bar between) of cyclothiazide (CTZ, 90 μM), CX546 (90 μM), or a control of 0.1% DMSO, which served as carrier for both drugs. Symbols represent mean±standard deviation (SD). Formal in-treatment statistical comparisons for post-protocol time points to their pre-protocol control are noted by marks of significance (n.s. not significant, *p≦0.05, **p≦0.01, ***p≦0.001). As shown by FIG. 2, a short-duration application of CTZ can induce between a 200-300% increase of ∫XII nerve output.

To look at the dose-response characteristics, cyclothiazide was applied in varying concentrations to the neonatal medullary slice preparation. FIG. 3 summarizes group data for the percent increase of integrated hypoglossal (∫XII). Group data showing normalized ∫XII nerve amplitude at 1 hour following a 1 hour application of 3, 9, 30, or 90 μM cyclothiazide. Symbols represent mean±SD of control. According to FIG. 3, ∫XII nerve amplitude increases as a function of cyclothiazide concentration. These data indicate that the level of effect may be titrated as necessary for the treatment of different diseases.

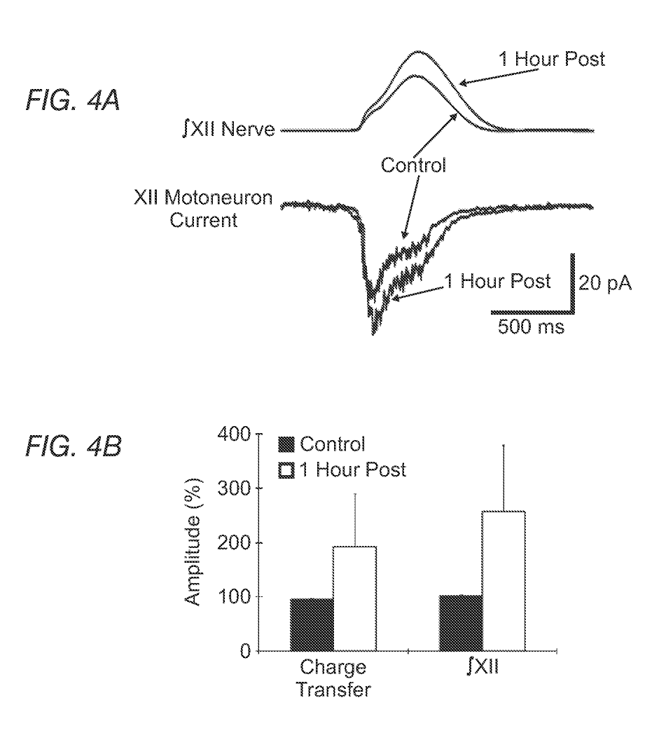

FIG. 4 shows cyclothiazide potentiates endogenously generated XII motoneuron drive currents. FIG. 4A shows ∫XII nerve burst and motoneuron currents from control period and 1 hour post-treatment with cyclothiazide (90 μM for 15 minutes). According to FIG. 4A, treatment with cyclothiazide induces ∫XII nerve burst and motoneuron drive current. FIG. 4B summarizes group data comparing XII motoneuron charge transfer and ∫XII nerve peak amplitude between control period and 1 hour post-treatment. According to FIG. 4B, cyclothiazide treatment induces XII motoneuron charge transfer and increase of ∫XII nerve amplitude. Therefore, cyclothiazide-induced enhancement of motoneuronal currents underlies enhanced XII nerve discharge.

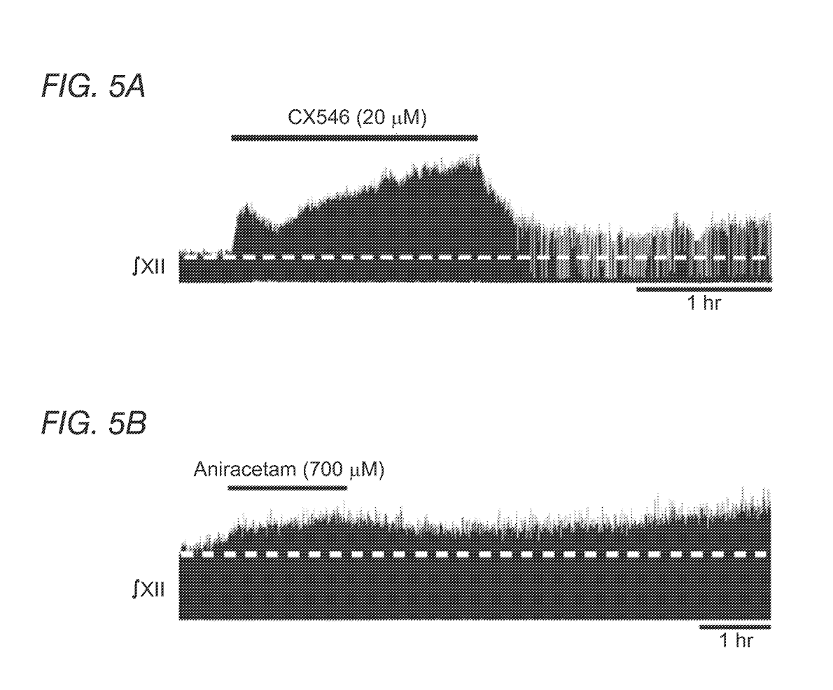

CX546 and aniracetam have similar but attenuated effects on motor activity in the neonatal medullary slice. FIG. 5 is a measurement of integrated hypoglossal (∫XII) nerve recordings showing the effects of bath application of CX546 or aniracetam. Traces show integrated hypoglossal nerve recordings (∫XII) from neonatal rat medullary slices exposed by bath application of CX546 (20 μM) (FIG. 5A) and aniracetam (700 μM) (FIG. 5B), a structural antecedent to CX546. According to FIG. 5, longer-term (>2 hours) application of lower doses of CX546 or higher doses of aniracetam from which CX546 is derived induces a persistent potentiation of ∫XII nerve discharge amplitude. Both agents induce a rapid increase in ∫XII amplitude that persists after removal of the drug.

### Example 2

### In Vivo Animal Model

The experiments described below demonstrate that the effects of AMPA receptor modulators observed in the in vitro medullary slice experiments also occur in vivo. Anesthetized freely breathing rats provide a live animal model for determining effects of AMPA receptor modulators on genioglossus (GG) muscle activity.

Adult Sprague-Dawley rats (˜300 g) were anaesthetized with ketamine (100 mg kg⁻¹) and xylazine (10 mg kg⁻¹) injected intraperitoneally. 1-1.5 vol % isoflurane in O₂ was inhaled during the experimental stage to provide for stable recordings and continued anesthesia. To measure GG electromyographic activity (EMG), stainless steel wire electrodes were sutured into the muscle. The raw GG EMG signal was rectified filtered and integrated.

Respiratory frequency and tidal volume were measured via pressure transducer attached to the rostral end of a polyethylene cannula that was inserted into the trachea of the anesthetized rat. The animal was freely breathing without the use of any ventilator or other method of respiratory support. These methods were taken in part from those reported in Tan, W., et al. (2008) Nat. Neurosci. 11(5):538-40 and Janczewski, W. A. and Feldman, J. L. (2006) J. Physiol. 570(Pt 2):407-20, incorporated by reference in their entirety.

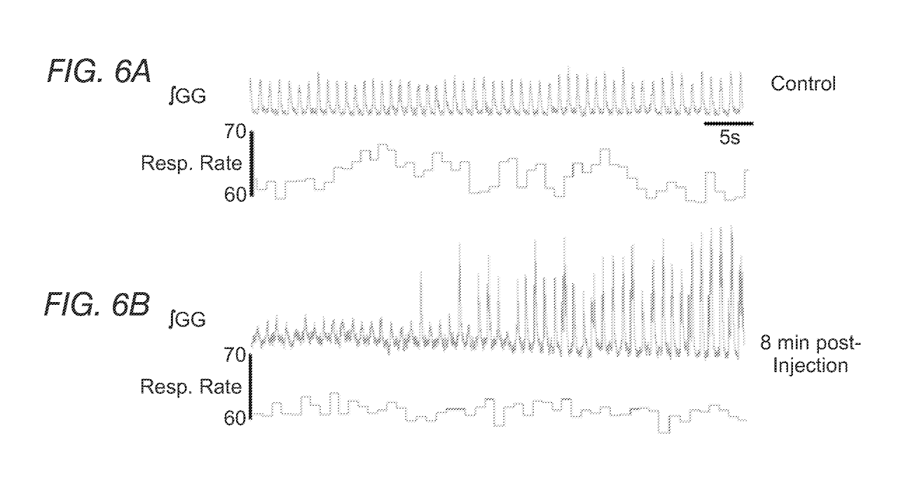

FIG. 6 is a measurement of integrated genioglossus (∫GG) muscle output and respiratory frequency in an anesthetized rat prior to application and after application of cyclothiazide to the hypoglossal (XII) motor nucleus. Cyclothiazide was applied by micropipette injection into the hypoglossal (XII) motor nucleus of an anesthetized rat. FIG. 6A, upper trace, shows control (prior to injection) integrated genioglossus muscle recording (∫GG), and lower trace, respiratory rate histogram prior to focal application of cyclothiazide. FIG. 6B, upper trace, shows post-injection ∫GG activity, and lower trace, respiratory rate histogram 8 minutes after focal application of cyclothiazide. Comparing the upper traces from FIGS. 6A and 6B shows that injection of cyclothiazide into the hypoglossal (XII) motor nucleus induces an increase in ∫GG activity as shown by the amplitude. Furthermore, comparing the lower traces of FIGS. 6A and 6B shows that injection of cyclothiazide into the hypoglossal (XII) motor nucleus does not affect respiratory frequency. These data demonstrate that focal microinjection of cyclothiazide potentiates the genioglossus muscle output without effecting respiratory rate.

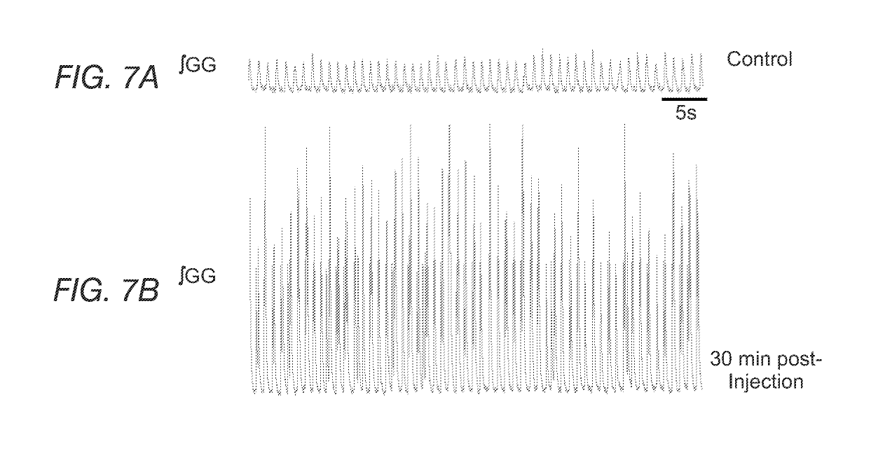

FIG. 7 is a measurement of ∫GG muscle activity and respiratory rate in the same anesthetized rat, freely breathing rat as in FIG. 6 for control and 30 minutes after application of cyclothiazide to the XII motor nucleus. FIG. 7A shows control ∫GG activity. FIG. 7B shows integrated genioglossus muscle recording (∫GG) 30 minutes post-cyclothiazide injection (1 μL, 25 mM). Quantification and comparison of ∫GG activity in FIGS. 7A and 7B revealed that application of cyclothiazide induced a 600% peak increase and 300% average increase of ∫GG activity relative to control. These data demonstrate that focal application of cyclothiazide in the hypoglossal nucleus induces long-lasting potentiation of genioglossal muscle activity in vivo.

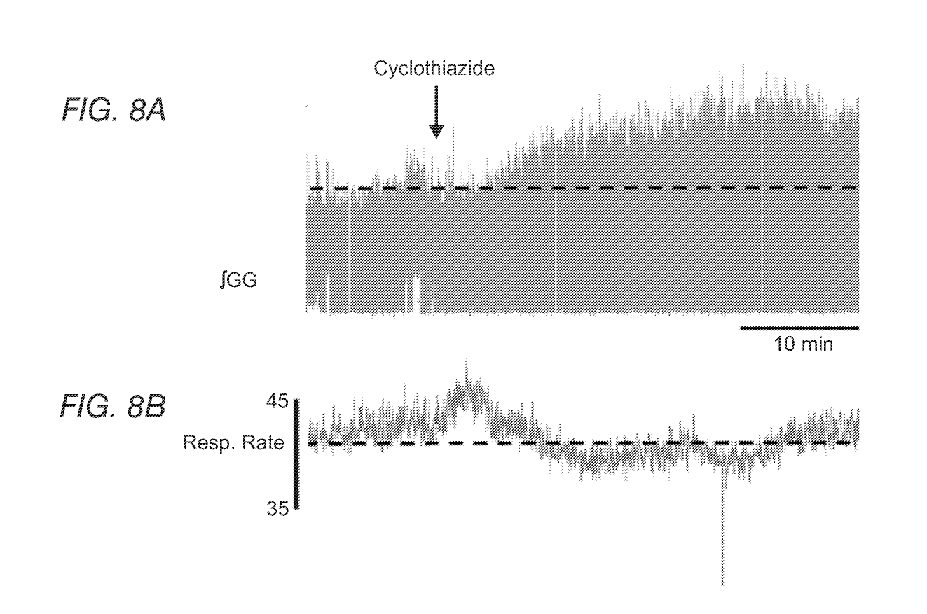

FIG. 8 shows ∫GG activity (FIG. 8A) and is a respiratory rate (FIG. 8B) in an anesthetized, freely breathing rat prior to and after applying cyclothiazide to the IV^{th} ventricle. Local application of cyclothiazide (time of application indicated by arrow) amplified genioglossus muscle activity. Elevation of ∫GG activity lasted ˜90 minutes, i.e., until the end of the experiment. Quantification of the ∫GG activity in FIG. 8A revealed that application of cyclothiazide induced a 150% peak increase and 50% average increase of ∫GG activity compared to control. These data show that application of cyclothiazide to the IV^{th} ventricle potentiates genioglossus muscle output in vivo and the long-lasting potentiation of ∫GG activity occurs without affecting respiratory rate. These data demonstrate that focal application of cyclothiazide to the IV^{th} ventricle induces long-lasting potentiation of genioglossus muscle output in vivo.

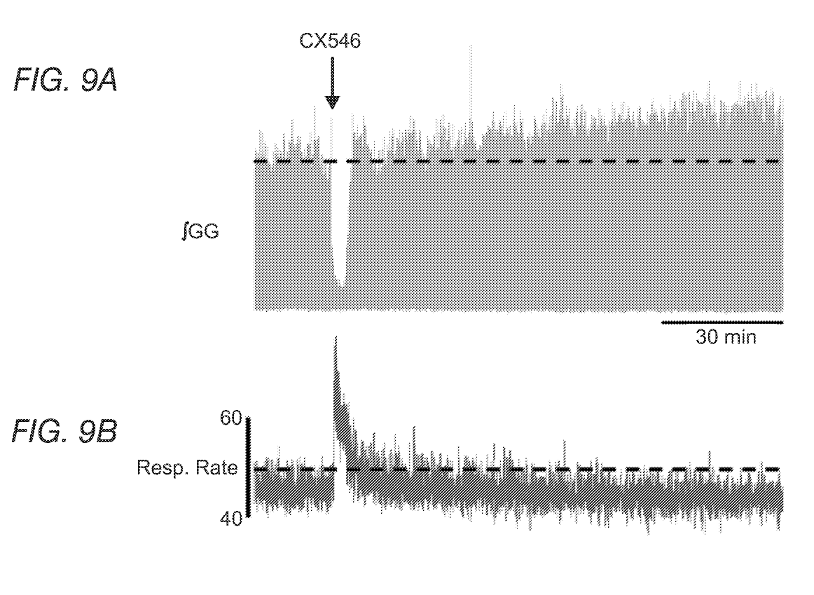

FIG. 9 shows ∫GG output (FIG. 9A) and respiratory rate (FIG. 9B) in an anesthetized rat prior to and after applying CX546 by intravenous injection. FIG. 9A shows intravenous injection of CX546 (arrow: 130 μg/kg) induces a long-lasting potentiation of ∫GG activity. FIG. 9B shows no long term changes in respiratory rate. These data demonstrate that systemic intravenous injection of CX546, which freely crosses the blood-brain barrier, into the femoral vein of a rat potentiates genioglossus muscle output in vivo. Quantification of ∫GG activity in FIG. 9A revealed that application of CX546 induced a 15% average increase of ∫GG activity compared to control. As shown in FIGS. 9A and 9B, genioglossus muscle activity remained elevated for more than 2 hours with no diminution of amplitude and no appreciable impact on respiration rate. The brief transient in respiratory rate was due to a rapid increase in blood pressure at the time of drug injection and could be repeated with injection of saline alone, while the changes in ∫GG output only occurred with injection of CX546.

In all in vivo experiments described above, no seizure activity or other neurological effects were observed in the rats studied, indicating the safety and efficacy of the AMPA receptor modulators at the doses given to increase genioglossus muscle activity.

### Example 3

Treatment of a Patient Having Obstructive Sleep Apnea with Benzothiadiazide

A patient is diagnosed as having obstructive sleep apnea. This diagnosis is confirmed by the presence of obstructive sleep apnea symptoms, including daytime sleepiness, unintentional sleep episodes, unrefreshing sleep, fatigue, or insomnia; awakening with breath holding, gasping, or choking; reports by a bed partner of loud snoring, breathing interruptions, or both in the patient's sleep; sleep study monitoring that documents >5 episodes of hypopnea and apnea per hour.

The patient is administered a pharmaceutical formulation of benzothiadiazide. Benzothiadiazide is administered to the patient orally or intravenously at a dosage of about 0.1 to 100 mg benzothiadiazide/kg body weight as determined by the attending physician. The patient is monitored for reduction or inhibition of obstructive sleep apnea and associated symptoms. It is discovered that treatment with benzothiadiazide reduces the symptoms of obstructive sleep apnea.

### Example 4

Treatment of a Patient Having Obstructive Sleep Apnea with Cyclothiazide

A patient is diagnosed as having obstructive sleep apnea. This diagnosis is confirmed by the presence of obstructive sleep apnea symptoms, including daytime sleepiness, unintentional sleep episodes, unrefreshing sleep, fatigue, or insomnia; awakening with breath holding, gasping, or choking; reports by a bed partner of loud snoring, breathing interruptions, or both in the patient's sleep; sleep study monitoring that documents >5 episodes of hypopnea and apnea per hour.

The patient is administered a pharmaceutical formulation of cyclothiazide. Cyclothiazide is administered to the patient intravenously at a dosage of about 0.1 to 100 mg cyclothiazide/kg body weight as determined by the attending physician. The patient is monitored for reduction or inhibition of obstructive sleep apnea and associated symptoms. It is discovered that treatment with cyclothiazide reduces the symptoms of obstructive sleep apnea.

### Example 5

Treatment of a Patient Having Obstructive Sleep Apnea with Aniracetam

A patient is diagnosed as having obstructive sleep apnea. This diagnosis is confirmed by the presence of obstructive sleep apnea symptoms, including daytime sleepiness, unintentional sleep episodes, unrefreshing sleep, fatigue, or insomnia; awakening with breath holding, gasping, or choking; reports by a bed partner of loud snoring, breathing interruptions, or both in the patient's sleep; sleep study monitoring that documents >5 episodes of hypopnea and apnea per hour.

The patient is administered a pharmaceutical formulation of aniracetam. Aniracetam is administered to the patient intravenously at a dosage of about 0.1 to 100 mg aniracetam/kg body weight as determined by the attending physician. The patient is monitored for reduction or inhibition of obstructive sleep apnea and associated symptoms. It is discovered that treatment with aniracetam reduces the symptoms of obstructive sleep apnea.

### Example 6

Treatment of a Patient Having Snoring with an AMPA Receptor Modulator

A patient is diagnosed as having snoring. This diagnosis is confirmed by the presence of snoring sounds made by the patient during sleep or conditions associated with snoring.

The patient is administered a pharmaceutical formulation of an AMPA receptor modulator. The AMPA receptor modulator is administered to the patient intravenously or orally at a dosage of about 0.1 to 100 mg AMPA receptor modulator/kg body weight as determined by the attending physician. The patient is monitored for reduction or inhibition of snoring and associated symptoms. It is discovered that treatment with AMPA receptor modulator reduces snoring.

### Example 7

Treatment of a Patient Having Snoring with Ampakine

A patient is diagnosed as having snoring. This diagnosis is confirmed by the presence of snoring sounds made by the patient during sleep or conditions associated with snoring.

The patient is administered a pharmaceutical formulation of an ampakine. The ampakine is administered to the patient intravenously or orally at a dosage of about 0.1 to 100 mg ampakine/kg body weight as determined by the attending physician. The patient is monitored for reduction or inhibition of snoring and associated symptoms. It is discovered that treatment with ampakine reduces snoring.

### Example 8

Treatment of a Patient Having Snoring with CX546

A patient is diagnosed as having snoring. This diagnosis is confirmed by the presence of snoring sounds made by the patient during sleep or conditions associated with snoring.

The patient is administered a pharmaceutical formulation of CX546. CX546 is administered to the patient intravenously or orally at a dosage of about 0.1 to 100 mg CX546/kg body weight as determined by the attending physician. The patient is monitored for reduction or inhibition of snoring and associated symptoms. It is discovered that treatment with CX546 reduces snoring.

### Example 9

Treatment of a Patient Having Snoring with Aniracetam

A patient is diagnosed as having snoring. This diagnosis is confirmed by the presence of snoring sounds made by the patient during sleep or conditions associated with snoring.

The patient is administered a pharmaceutical formulation of aniracetam. Aniracetam is administered to the patient intravenously or orally at a dosage of about 0.1 to 100 mg aniracetam/kg body weight as determined by the attending physician. The patient is monitored for reduction or inhibition of snoring and associated symptoms. It is discovered that treatment with aniracetam reduces snoring.

### Example 10

Treatment of a Patient Having Snoring with Benzothiadiazide

A patient is diagnosed as having snoring. This diagnosis is confirmed by the presence of snoring sounds made by the patient during sleep or conditions associated with snoring.

The patient is administered a pharmaceutical formulation of benzothiadiazide. Benzothiadiazide is administered to the patient intravenously or orally at a dosage of about 0.1 to 100 mg benzothiadiazide/kg body weight as determined by the attending physician. The patient is monitored for reduction or inhibition of snoring and associated symptoms. It is discovered that treatment with benzothiadiazide reduces snoring.

### Example 11

Treatment of a Patient Having Snoring with Cyclothiazide

A patient is diagnosed as having snoring. This diagnosis is confirmed by the presence of snoring sounds made by the patient during sleep or conditions associated with snoring.

The patient is administered a pharmaceutical formulation of cyclothiazide. Cyclothiazide is administered to the patient intravenously at a dosage of about 0.1 to 100 mg cyclothiazide/kg body weight as determined by the attending physician. The patient is monitored for reduction or inhibition of snoring and associated symptoms. It is discovered that treatment with cyclothiazide reduces snoring.

### Example 12

Treatment of a Patient Having Multiple Sclerosis with an AMPA Receptor Modulator

A patient is diagnosed as having multiple sclerosis confirmed by clinical standards for the diagnosis of multiple sclerosis.

The patient is administered a pharmaceutical formulation of an AMPA receptor modulator. The AMPA receptor modulator is administered to the patient orally or intravenously at a dosage of about 0.1 to 100 mg AMPA receptor modulator/kg body weight as determined by the attending physician. The patient is monitored for reduction or inhibition of symptoms associated with multiple sclerosis. It is discovered that treatment with an AMPA receptor modulator reduces or inhibits multiple sclerosis symptoms.

### Example 13

Treatment of a Patient Having Spinal Cord Injury with an AMPA Receptor Modulator

A patient is diagnosed as having spinal cord injury.

The patient is administered a pharmaceutical formulation of an AMPA receptor modulator. The AMPA receptor modulator is administered to the patient orally or intravenously at a dosage of about 0.1 to 100 mg AMPA receptor modulator/kg body weight as determined by the attending physician. The patient is monitored for improvement of sensory or motor function. It is discovered that treatment with an AMPA receptor modulator improves sensory or motor function.

The invention now being fully described, it will be apparent to one of ordinary skill in the art that many changes and modifications can be made thereto without departing from the spirit or scope of the appended claims.

1-35. (canceled)

36. A method of increasing genioglossus muscle tone in a subject having sleep-disordered breathing comprising administering to the subject an effective amount of an AMPA receptor modulator sufficient to increase genioglossus muscle activity.

37. The method of claim 36, wherein the AMPA receptor modulator is an ampakine.

38. The method of claim 37, wherein the ampakine is CX546.

39-41. (canceled)

42. The method of claim 37, wherein the effective amount of the ampakine sufficient to increase genioglossus muscle activity is also sufficient to reduce or inhibit snoring in the subject.

43. The method of claim 36, wherein the AMPA receptor modulator is a benzothiadiazide.

44. The method of claim 43, wherein the benzothiadiazide is cyclothiazide.

45-47. (canceled)

48. The method of claim 43, wherein the effective amount of the benzothiadiazide sufficient to increase genioglossus muscle activity is also sufficient to reduce or inhibit snoring in the subject.

49. The method of claim 43, wherein the effective amount of the benzothiadiazide sufficient to increase genioglossus muscle activity is also sufficient to reduce or inhibit obstructive sleep apnea in the subject.

50. The method of claim 36, wherein the AMPA receptor modulator is aniracetam.

51. The method of claim 50, wherein the effective amount of aniracetam sufficient to increase genioglossus muscle activity is also sufficient to reduce or inhibit snoring in the subject.

52. The method of claim 50, wherein the effective amount of aniracetam sufficient to increase genioglossus muscle activity is also sufficient to reduce or inhibit obstructive sleep apnea in the subject.

53. A method of treating snoring in a subject having snoring comprising administering to the subject an effective amount of an AMPA receptor modulator sufficient to reduce or inhibit snoring.

54. The method of claim 53, wherein the AMPA receptor modulator is an ampakine.

55. The method of claim 54, wherein the ampakine is CX546.

56. The method of claim 53, wherein the AMPA receptor modulator is a benzothiadiazide.

57. The method of claim 56, wherein the benzothiadiazide is cyclothiazide.

58. The method of claim 53, wherein the AMPA receptor modulator is aniracetam.

59. A method of treating obstructive sleep apnea in a subject having obstructive sleep apnea comprising administering to the subject an effective amount of a benzothiadiazide sufficient to reduce or inhibit obstructive sleep apnea.

60. The method of claim 59, wherein the benzothiadiazide is cyclothiazide.

61. A method of treating obstructive sleep apnea in a subject comprising administering to the subject an effective amount of aniracetam sufficient to reduce or inhibit obstructive sleep apnea.

62-100. (canceled)
